(19) United States
(12) Patent Application Publication
(10) Pub. No.: US 2017/0212073 A1
(43) Pub. Date: Jul. 27, 2017

(54) Method For Operating A Gas Sensor For Improving The Detection Of Nitrogen Oxides

(71) Applicant: Siemens Aktiengesellschaft, Muenchen (DE)

(72) Inventors: Sabine Fischer, Ludwigsburg (DE); Maximilian Fleischer, Hoehenkirchen (DE); Erhard Magori, Feldkirchen (DE); Ralf Moos, Bayreuth (DE); Roland Pohle, Herdweg (DE)

(73) Assignee: Siemens Aktiengesellschaft, Muenchen (DE)

(21) Appl. No.: 15/326,714

(22) PCT Filed: Jul. 21, 2015
(86) PCT No.: PCT/EP2015/066641

§ 371 Date: Jan. 17, 2017

(30) Foreign Application Priority Data
Jul. 23, 2014 (DE) 102014214409.4

(51) Int. Cl. G01N 27/406; G01N 27/407; G01N 33/00

(52) U.S. Cl. CPC G01N 27/4067; G01N 33/0037; G01N 27/4074; G01N 27/4075

(57) ABSTRACT

A gas sensor for detecting nitrogen oxides in a gas mixture may include at least two electrodes of the same material arranged on an oxygen ion conductor. When the gas sensor is operated, both electrodes come into contact with the gas mixture. The gas sensor is then heated from a first temperature to a second temperature. The second temperature of the gas sensor is maintained for a maximum of 15 minutes. The gas sensor is then cooled from the second temperature to the first temperature. During the heating, maintaining and/or cooling of the temperature, a cyclical polarisation is performed with alternating polarity including a polarisation voltage below the reduction voltage of the oxygen ion conductor.

### CROSS-REFERENCE TO RELATED APPLICATIONS

This application is a U.S. National Stage Application of International Application No. PCT/EP2015/066641 filed Jul. 21, 2015, which designates the United States of America, and claims priority to DE Application No. 10 2014 214 409.4 filed Jul. 23, 2014, the contents of which are hereby incorporated by reference in their entirety.

### TECHNICAL FIELD

The invention relates to a method for operating a gas sensor for improving the long-term stability of the gas sensor.

### BACKGROUND

Increasing demands with regard to the emissions of exhaust gases and the efficiency during the operation of power plants, combustion plants, waste incineration plants, gas turbines and engines of all types can also be addressed inter alia through determination of the composition of gases in the respective installations during ongoing operation, and evaluation of said composition for improved operation. This results in a need for sensors for determining components of a gas mixture. One example for this is the ever-increasing number of motor vehicles, for which ever more stringent exhaust-gas regulations must at the same time be complied with in order to limit the damage to the environment and to health caused by combustion exhaust gases. Aside from harmful exhaust-gas components such as sulfur oxides and carbon dioxide, the group of nitrogen oxides, referred to as NOx for short, is taking on ever greater significance. Enormous technical and financial outlay is being invested into reducing the nitrogen oxide emissions. Examples for this are exhaust-gas recirculation and selective catalytic reduction. In order to monitor the function of said processes and to lower the operating costs, ongoing monitoring of the NOx concentration in the exhaust gas of the vehicle is necessary.

Specifically in automotive applications, it is prescribed in certain countries that the functionality of the exhaust-gas aftertreatment be diagnosed in the vehicle itself. Automobile manufacturers must ensure that a randomly selected vehicle complies with the emissions regulations even after a long operating time. In the case of diesel vehicles in particular, the monitoring of NOx storage catalytic converters and SCR (selective catalytic reduction) catalytic converters for reducing the NOx emissions is a task which is being worked upon intensively.

Known sensors for the measurement of NOx are optical or chemoluminescence-based systems. Aside from the high price, said systems have the disadvantage that an extractive measurement is necessary, that is to say an extraction of gas is necessary. For many applications, this is associated with high outlay.

Known sensors that overcome these disadvantages are based on yttrium-stabilized zirconium dioxide (YSZ) and are similar in construction to the conventional lambda probe. Here, use is made of electrodes composed of the same material, for example platinum. In the case of the classic functional principle of said sensor, a simultaneous measurement of oxygen and NOx is performed in a two-chamber system. Disadvantages that remain in the case of this typical sensor principle are however a complex construction of the sensor and thus a high price. A second, relatively new possibility for a functional principle is disclosed in the German patent application 102013222195.9. Said gas sensor comprises an oxygen-ion-conducting material and at least two electrodes arranged on the ion-conducting material. In the case of said gas sensor, the electrodes are composed of the same material. The gas sensor is furthermore designed such that, during operation of the gas sensor, both electrodes come into contact with the gas mixture. In this case, it is not necessary for the gas sensor to have a two-chamber system. This greatly simplifies the construction of the gas sensor. The measurement of said gas sensors is based on a polarization method in which the NOx concentration is measured by way of voltage pulses and a subsequent depolarization. Said sensors disadvantageously exhibit a change both in the polarization current and in the discharge curves in the case of relatively long-term use. Said change results in a significant degradation of the sensor signal. Said degradation is highly dependent on the voltage amplitude with which the sensor is operated, and on the polarization currents that have been measured during the voltage pulses. Said gas sensor thus disadvantageously degrades considerably more quickly at high temperatures, and in association therewith in the presence of relatively high currents, than at moderate temperatures and low voltage amplitudes.

### SUMMARY

One embodiment provides a method for operating a gas sensor for the detection of nitrogen oxides in a gas mixture, having the following steps: providing a gas sensor having at least two electrodes composed of the same material arranged on an oxygen-ion conductor, wherein, during operation of the gas sensor, both electrodes come into contact with the gas mixture, heating the gas sensor from a first temperature to a second temperature, maintaining the second temperature of the gas sensor for at most fifteen minutes, and cooling the gas sensor from the second temperature to the first temperature, wherein, during the heating, the maintaining and/or the cooling of the temperature, a cyclic polarization with alternating polarity is performed with a polarization voltage below the reduction voltage of the oxygen-ion conductor.

In one embodiment, the oxygen-ion conductor comprises yttrium-stabilized zirconium dioxide.

In one embodiment, the gas sensor is surrounded by an oxygen-containing atmosphere.

In one embodiment, the polarization voltage is less than 2.3 V.

In one embodiment, the polarization voltage lies in a range between 0.5 V and 1 V.

In one embodiment, the polarity of the cyclic polarization alternates with a frequency of at least 0.5 Hz.

In one embodiment, the polarity of the cyclic polarization alternates with a frequency in a range from 0.5 Hz to 1.0 Hz.

In one embodiment, the heating and the cooling are performed at a rate in a range from 1 K/min to 20 K/min.

In one embodiment, the first temperature amounts to at least 350° C.

In one embodiment, the second temperature amounts to at most 1200° C.

### BRIEF DESCRIPTION OF THE DRAWINGS

Example aspects and embodiments of the invention are described in detail below with reference to the drawings, in which:

FIG. 1 shows two NO characteristic curves of the gas sensor after 8 h and 40 h of operation without pretreatment;

FIG. 2 shows a temperature profile and possible polarization sections during a pretreatment of the gas sensor;

FIG. 3 shows four NO characteristic curves of the gas sensor according to the first measurement, after 50 h, 100 h and 150 h, following a pretreatment, and

FIG. 4 shows a comparison of a sensor signal without pretreatment and with pretreatment.

### DETAILED DESCRIPTION

Embodiments of the present invention provide a method which may overcome at least some of the stated disadvantages and which may provide a gas sensor with improved long-term stability of.

One embodiment provides a method for operating a gas sensor for the detection of nitrogen oxides in a gas mixture. A gas sensor having at least two electrodes composed of the same material arranged on an oxygen-ion conductor is provided. During operation of the gas sensor, both electrodes come into contact with the gas mixture. Subsequently, the gas sensor is heated from a first temperature to a second temperature. The second temperature of the gas sensor is maintained for at most 15 minutes. Subsequently, the gas sensor is cooled from the second temperature to the first temperature. During the heating, the maintaining and/or the cooling of the temperature, a cyclic polarization with alternating polarity is performed with a polarization voltage below the reduction voltage of the oxygen-ion conductor.

The method may improve the long-term stability and sensitivity of the sensor, as compared with known sensors. In experiments, it has been found that, whereas the signal strength of the sensor without pretreatment has degraded significantly after just 40 operating hours, it was possible to significantly improve this behavior in the case of the pretreated sensor. Aside from a classic pretreatment of a gas sensor, it is likewise conceivable for said activation process to be performed a further time, that is to say as a reactivation by way of the disclosed method. Furthermore, by way of the advantageously low polarization voltage below the reduction voltage of the oxygen-ion conductor, it is ensured that the oxygen-ion conductor is neither reduced nor changed. Said low voltage also has the result that a cyclic reactivation can also be performed repeatedly if the sensor signal has diminished during the operation by way of the voltage pulse method.

In one embodiment, the gas sensor is surrounded by an oxygen-containing atmosphere. This may advantageously prevent a reducing atmosphere from forming around the oxygen-ion conductor.

In a further embodiment, the oxygen-ion conductor comprises yttrium-stabilized zirconium dioxide. The polarization voltage is then advantageously less than 2.3 V. In some embodiment, the polarization voltage lies in a range between 0.5 V and 1 V. Said polarization voltages may advantageously prevent a reduction of the oxygen-ion conductor, so-called blackening.

In a further embodiment, the polarity of the cyclic polarization alternates with a frequency of at least 0.5 Hz. In some embodiments, the cyclic polarization may alternate with a frequency in a range from 0.5 Hz to 1 Hz.

In a further embodiment, the heating and the cooling are performed at a rate in a range from 1 K/min to 20 K/min.

In a further embodiment, the first temperature is at least 350° C. The second temperature is at most 1200° C.

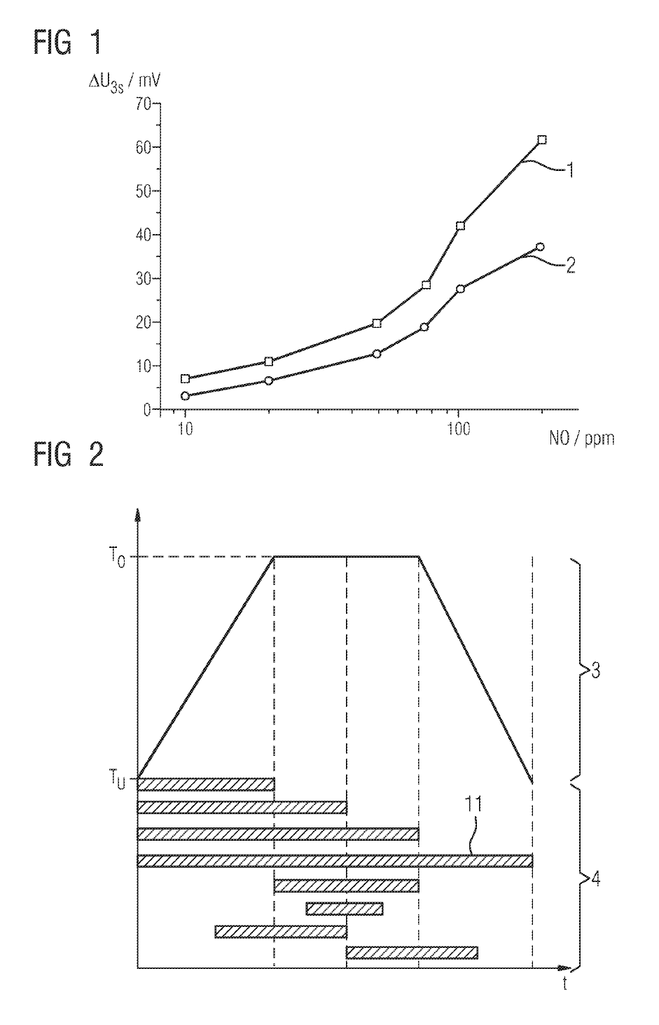

The NO characteristic curves illustrated in FIG. 1 show the voltage differences of the sensor signal after a discharging time of 3 s versus an increasing concentration of nitrogen monoxide. The first NO characteristic curve 1 was measured after an operating time of the gas sensor of 8 h. The second NO characteristic curve 2 was measured after an operating time of the gas sensor of 40 h. The gas sensor is operated at a temperature of 350° C. Said gas sensor comprises yttrium-stabilized zirconium dioxide (YSZ) as oxygen-ion conductor. Furthermore, said gas sensor comprises two platinum electrodes which are connected to a device for generating and measuring voltage U. Said gas sensor is inserted into a space with the gas mixture to be measured. Before the operation, the gas sensor has not been pretreated by way of a method for ensuring the long-term stability of the gas sensor. A comparison of the two NO characteristic curves clearly shows that the voltage differences decrease significantly after 40 h of operation. In the case of high NO concentrations, the voltage differences disadvantageously decrease even by ⅔ of the original difference.

To counteract this effect, the method shown in FIG. 2 for the operation of the gas sensor is carried out. FIG. 2 shows a typical temperature profile 3 and different possible polarization time periods 4. The time t is plotted on the x axis. Here, the temperature of the gas sensor is typically heated from the lower temperature T_{U} of 420° C. to an upper temperature T_{O} of 800° C. The heating is performed at a heating rate of 10 K/min, and thus lasts for 38 minutes. The maximum temperature is maintained for 5 minutes, and subsequently, the temperature is lowered from T_{O} to T_{U} again, wherein the cooling rate is likewise 10 K/min. In this example, the polarization takes place over the entire time of the temperature profile, illustrated by the first polarization time period 11. It is likewise possible for the alternating cyclic polarization to be performed in each case only in the sections shown in FIG. 2. In this example, the voltage amplitude is at most 1 V. The polarization is performed by way of a rectangular function, in which the voltage is constant for a time duration t₀. Polarizations with sinusoidal functions or further functions are likewise conceivable. The frequency of the alternating voltage amounts to between 0.5 Hz and 1 Hz, wherein, in this example, said frequency is 0.7 Hz. Throughout operation, the sensor is situated in an oxygen-containing atmosphere in order to prevent reduction of the YSZ.

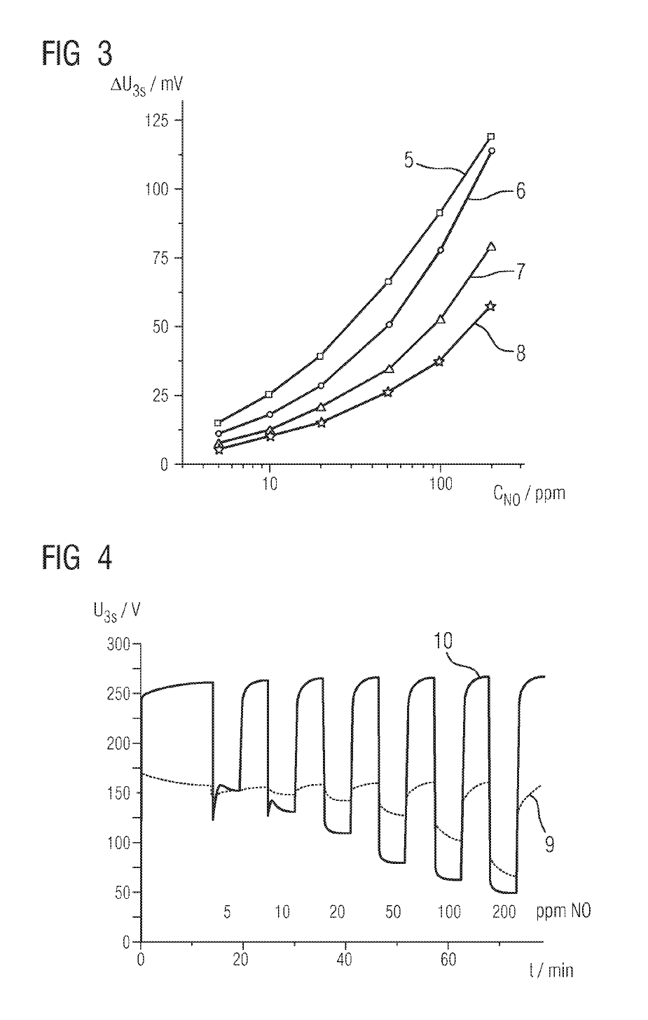

FIG. 3 illustrates NO characteristic curves for a sensor that has been operated with alternating polarization, after different lengths of time. As in FIG. 1, it is the case in FIG. 3 that the voltage differences of the sensor signal after a discharging time of 3 s are plotted versus an increasing concentration of nitrogen monoxide. Even in the case of the first measurement, a considerable increase in the voltage difference is evident for all carbon monoxide concentrations. For example, in the case of an NO concentration of 200 ppm, a voltage difference of 100 mV is measured at an operating time of 50 h. Without a pretreatment of the gas sensor, said value was 35 mV after 40 h of operation. Even in the case of very low NO concentrations, said increase of the voltage difference is considerable. Thus, from just 5 ppm NO upward, the measurement accuracy of the gas sensor increases considerably after the pretreatment. Even after 150 h of operation, the voltage difference is still so great that the gas sensor performs reliable measurement. The long-term stability of the gas sensor, and the sensitivity thereof, have thus been improved.

FIG. 4 likewise clearly shows the improvement in the measurement signal. The first sensor signal 9 versus the time exhibits only small voltage differences in particular in the case of low nitrogen monoxide concentrations. After the pretreatment, the second sensor signal 10 exhibits a considerable voltage difference even in the presence of low NO concentrations, such that reliable detection is realized from concentrations of just 5 ppm NO upward.

What is claimed is:

1. A method for operating a gas sensor for detecting nitrogen oxides in a gas mixture, the method comprising:
providing a gas sensor having at least two electrodes composed of the same material arranged on an oxygen-ion conductor, wherein during operation of the gas sensor, the at least two electrodes come into contact with the gas mixture,
heating the gas sensor from a first temperature to a second temperature,
maintaining the second temperature of the gas sensor for at most fifteen minutes,
cooling the gas sensor from the second temperature to the first temperature, and
during at least one of the heating step, the maintaining step, or the cooling step, performing a cyclic polarization with alternating polarity using a polarization voltage below the reduction voltage of the oxygen-ion conductor.

2. The method of claim 1, wherein the oxygen-ion conductor comprises yttrium-stabilized zirconium dioxide.

3. The method of claim 1, wherein the gas sensor is surrounded by an oxygen-containing atmosphere.

4. The method of claim 2, wherein the polarization voltage is less than 2.3 V.

5. The method of claim 4, wherein the polarization voltage lies in a range between 0.5 V and 1 V.

6. The method of claim 1, wherein the polarity of the cyclic polarization alternates with a frequency of at least 0.5 Hz.

7. The method of claim 6, wherein the polarity of the cyclic polarization alternates with a frequency in a range from 0.5 Hz to 1.0 Hz.

8. The method of claim 1, wherein the heating and the cooling are performed at a rate in a range between 1 K/min and 20 K/min.

9. The method of claim 1, wherein the first temperature is at least 350° C.

10. The method of claim 1, wherein the second temperature is at most 1200° C.
